(19) United States
(12) Patent Application Publication
(10) Pub. No.: US 2024/0310279 A1
(43) Pub. Date: Sep. 19, 2024

(54) BIOLOGICAL INFORMATION MEASUREMENT APPARATUS AND BIOLOGICAL INFORMATION MEASUREMENT SYSTEM

(71) Applicant: SEIKO EPSON CORPORATION, TOKYO (JP)

(72) Inventor: Ayae SAWADO, Kai-shi (JP)

(21) Appl. No.: 18/607,425

(22) Filed: Mar. 16, 2024

(30) Foreign Application Priority Data
Mar. 17, 2023 (JP) 2023-042746

(51) Int. Cl. G01N 21/49; G01N 21/59; G16B 40/10

(52) U.S. Cl. CPC G01N 21/49; G01N 21/59; G16B 40/10

(57) ABSTRACT

A biological information measurement apparatus includes: a light emitting unit configured to emit laser light; a light receiving unit configured to receive scattered light generated when the laser light enters the living body; a case accommodating the light emitting unit and the light receiving unit; and a light transmission member attached to the case at a position facing the living body. The light transmission member has a first surface where the laser light enters and a second surface where the laser light entering the first surface enters, and D1<D2, in which a parallel axis parallel to an intersection line between the first surface and an entering surface on which the light emitting unit and the light receiving unit are disposed is defined as a first axis, an orthogonal axis orthogonal to the first axis on the first surface is defined as a second axis, D1 is a first diameter of the laser light along the first axis, and D2 is a second diameter of the laser light along the second axis.

The present application is based on, and claims priority from JP Application Serial Number 2023-042746, filed Mar. 17, 2023, the disclosure of which is hereby incorporated by reference herein in its entirety.

### BACKGROUND

### 1. Technical Field

The present disclosure relates to a biological information measurement apparatus and a biological information measurement system.

### 2. Related Art

Biological information measurement apparatuses that emit laser light to a living body are known. JP-A-2022-144578 discloses a biological information acquisition apparatus as an example of the biological information measurement apparatus. JP-A-2022-144578 describes a biological information acquisition apparatus that causes a light beam transmitted through a light branching element to enter a living body. The light branching element is made of a light transmission material. The light branching element is attached to a hole of a case as an example. The light branching element is disposed so as to be contactable with an inspection site of a living body. The light branching element is provided as a cover glass.

JP-A-2022-144578 is an example of the related art.

The light transmission material reflects the laser light by a front surface and a back surface. Interference light may be generated by front surface reflected light reflected by the front surface and back surface reflected light reflected by the back surface. The interference light decreases measurement accuracy of the biological information.

### SUMMARY

A biological information measurement apparatus according to an aspect of the present disclosure includes: a light emitting unit configured to emit laser light to a living body; a light receiving unit configured to receive scattered light generated when the laser light enters the living body; a case accommodating the light emitting unit and the light receiving unit; and a light transmission member attached to the case at a position facing the living body. The light transmission member has a first surface where the laser light enters and a second surface where the laser light entering the first surface enters, and D1<D2, in which a parallel axis parallel to an intersection line between the first surface and an entering surface on which the light emitting unit and the light receiving unit are disposed is defined as a first axis, an orthogonal axis orthogonal to the first axis on the first surface is defined as a second axis, D1 is a first diameter of the laser light along the first axis, and D2 is a second diameter of the laser light along the second axis.

A biological information measurement system according to an aspect of the present disclosure includes: a biological information measurement apparatus including a light emitting unit configured to emit laser light to a living body, a light receiving unit configured to receive scattered light generated when the laser light enters the living body and generate a detection signal, a case accommodating the light emitting unit and the light receiving unit, a light transmission member attached to the case at a position facing the living body, and a communication unit configured to transmit the detection signal; and a control apparatus including a terminal communication unit configured to receive the detection signal, and an analysis unit configured to analyze biological information of the living body using the detection signal. The light transmission member has a first surface where the laser light enters and a second surface where the laser light entering the first surface enters, and D1<D2, in which a parallel axis parallel to an intersection line between the first surface and an entering surface on which the light emitting unit and the light receiving unit are disposed is defined as a first axis, an orthogonal axis orthogonal to the first axis on the first surface is defined as a second axis, D1 is a first diameter of the laser light along the first axis, and D2 is a second diameter of the laser light along the second axis.

### BRIEF DESCRIPTION OF THE DRAWINGS

FIG. 1 is a diagram showing a schematic configuration of a measurement system.

FIG. 2 is a diagram showing a schematic configuration of a measurement surface of a measurement apparatus.

FIG. 3 is a diagram showing a block configuration of the measurement apparatus.

FIG. 4 is a diagram showing a schematic configuration of laser light emitted from a light emitting element unit.

FIG. 5 is a diagram showing an outline of an optical measurement performed by a detection unit.

FIG. 6 is a diagram showing an enlarged configuration of reflected light near a cover glass.

FIG. 7 is a diagram showing a schematic configuration of a spot on a front surface of the cover glass.

FIG. 8 is a diagram showing an enlarged configuration of the reflected light near the cover glass.

FIG. 9 is a diagram showing a block configuration of the measurement system.

### DESCRIPTION OF EMBODIMENTS

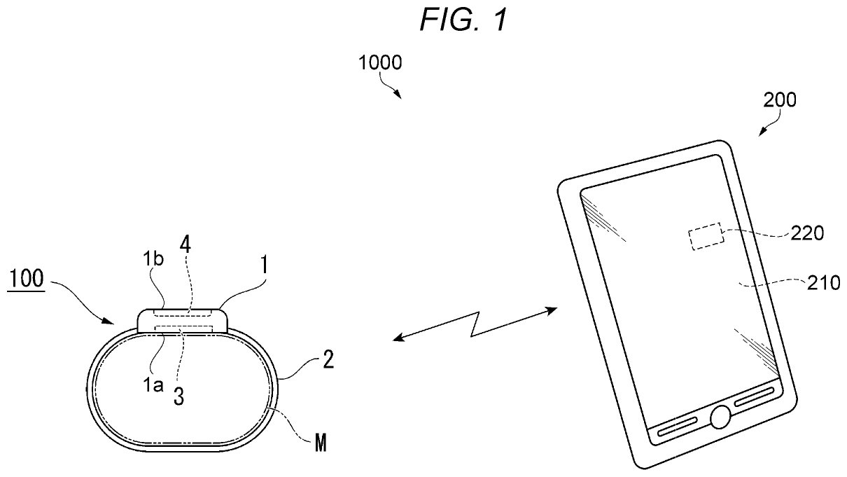

FIG. 1 shows a schematic configuration of a measurement system 1000. The measurement system 1000 measures a blood flow, a blood volume, a blood flow rate, a pulse, and the like. The measurement system 1000 evaluates a heart rate, a blood pressure, and the like using a measurement result. As an example, the measurement system 1000 evaluates the blood pressure or the like using the blood flow as an index. The measurement system 1000 corresponds to an example of a biological information measurement system.

The measurement system 1000 includes a measurement apparatus 100 and a tablet terminal 200. The measurement apparatus 100 and the tablet terminal 200 are communicably connected. The measurement system 1000 shown in FIG. 1 performs wireless communication connection between the measurement apparatus 100 and the tablet terminal 200. The connection between the measurement apparatus 100 and the tablet terminal 200 is not limited to wireless. The measurement apparatus 100 and the tablet terminal 200 may be communicably connected by a wire. The measurement apparatus 100 corresponds to an example of a biological information measurement apparatus. The tablet terminal 200 corresponds to an example of a control apparatus.

The measurement apparatus 100 measures biological information or various types of data related to the biological information of a user M such as a human. The user M corresponds to an example of a living body. The measurement apparatus 100 shown in FIG. 1 is a wristwatch type portable device worn by the user M at a measurement site. The measurement apparatus 100 is worn on a wrist of the user M. The measurement apparatus 100 is not limited to the wristwatch type. A form of the measurement apparatus 100 is not limited as long as the measurement apparatus 100 can be worn on the user M. The measurement apparatus 100 measures a blood flow, a blood volume, a blood flow rate, a pulse, and the like over time. The measurement apparatus 100 may measure biological information other than the blood flow, and the like. The measurement apparatus 100 measures, for example, a blood oxygen saturation concentration and a skin perfusion pressure. The measurement apparatus 100 includes a housing 1 and a belt 2. The housing 1 accommodates a detection unit 3 and a display unit 4.

The housing 1 is an exterior accommodating units and the like provided in the measurement apparatus 100. The housing 1 has a measurement surface 1a and a display surface 1b. The measurement surface 1a is a surface facing the measurement site of the user M. At least a part of the measurement surface 1a comes into contact with the measurement site of the user M. The display surface 1b is a surface that can be visually recognized by the user M. In addition to the detection unit 3 and the display unit 4, the housing 1 accommodates a control unit 30, a memory 40, and the like to be described later. The housing 1 corresponds to an example of a case.

The belt 2 is a member that is used when the user M wears the housing 1 at the measurement site. The belt 2 is attached to, for example, a side surface of the housing 1. The belt 2 is wound around the measurement site, such that the housing 1 is worn by the user M at the measurement site. The measurement apparatus 100 shown in FIG. 1 includes the belt 2, but is not limited thereto. The measurement apparatus 100 may not include the belt 2. The measurement apparatus 100 may be worn at a chest, an arm, or the like of the user M with a tape or the like. The measurement apparatus 100 is preferably wound around the measurement site of the user M using the belt 2. When the user M wears the measurement apparatus 100 at the measurement site with the belt 2, an arrangement of a light emitting element unit 10 and a light receiving element unit 20 to be described later with respect to the user M is determined. The belt 2 corresponds to an example of a band.

The detection unit 3 is disposed on the measurement surface 1a of the housing 1. The detection unit 3 is disposed at a position facing the measurement site of the user M. The detection unit 3 acquires various types of data used when measuring the biological information.

The display unit 4 is disposed on the display surface 1b of the housing 1. The display unit 4 is formed to be capable of being visually recognized by the user M. The display unit 4 displays various types of measured biological information. The display unit 4 may display a reliability index of the biological information and information other than the biological information, such as a time. The display unit 4 may not be provided.

The tablet terminal 200 analyzes the biological information such as the blood flow. The tablet terminal 200 may calculate the biological information using various data acquired from the measurement apparatus 100. The tablet terminal 200 acquires various data transmitted from the measurement apparatus 100. The tablet terminal 200 calculates and analyzes the biological information using the various data. The measurement system 1000 shown in FIG. 1 includes the tablet terminal 200, but the present disclosure is not limited thereto. The measurement system 1000 may include a personal computer, a smartphone, a dedicated terminal for analyzing the biological information, or the like, instead of the tablet terminal 200. The tablet terminal 200 includes a display 210 and a terminal control unit 220.

The display 210 displays an analysis result of the biological information. The display 210 may display various measurement results such as a temporal change in blood flow. The display 210 may display the biological information such as the blood flow and the blood pressure in a chart manner.

The terminal control unit 220 analyzes the biological information such as the blood flow. The terminal control unit 220 acquires various data transmitted from the measurement apparatus 100. The terminal control unit 220 analyzes the biological information of the user M using the various data. The terminal control unit 220 generates chart data related to the biological information such as the blood flow and the blood pressure. The terminal control unit 220 controls the display 210 to display the analysis result of the various biological information and the biological information in the chart manner on the display 210. The terminal control unit 220 corresponds to an example of an analysis unit.

### First Embodiment

The first embodiment shows the measurement apparatus 100 that calculates the biological information in the measurement apparatus 100. The measurement apparatus 100 according to the first embodiment calculates the biological information using the light detection signal. The measurement apparatus 100 transmits the calculated biological information to the tablet terminal 200. The measurement apparatus 100 may analyze the biological information and display the analysis result on the display unit 4.

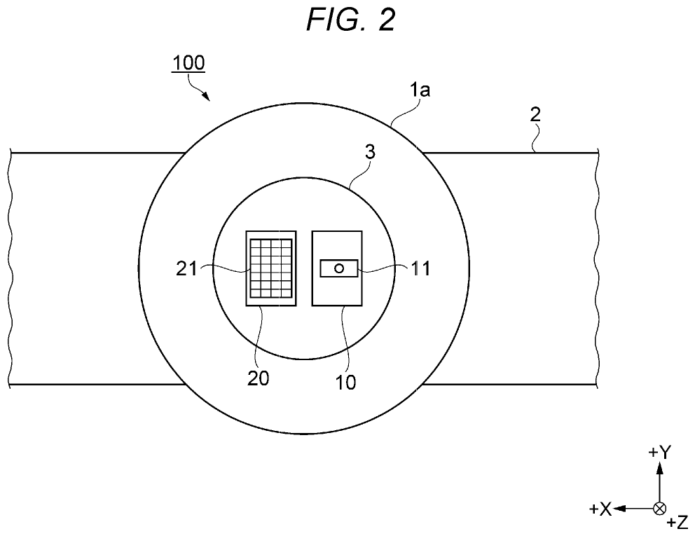

FIG. 2 shows a schematic configuration of the measurement surface 1a of the measurement apparatus 100. FIG. 2 shows a schematic configuration of the measurement surface 1a of the measurement apparatus 100 when viewed from a measurement site side of the user M. The measurement surface 1a shown in FIG. 2 is formed in a circular shape, but the shape is not limited to the circular shape. The measurement surface 1a may be formed in various shapes such as a square shape and an elliptical shape. The detection unit 3 is disposed on the measurement surface 1a. The detection unit 3 includes the light emitting element unit 10 and the light receiving element unit 20. The detection unit 3 may include a temperature detection sensor, a power terminal, and the like (not shown).

A plurality of drawings including FIG. 2 show an XYZ coordinate system. An X-axis is an axis parallel to a direction in which the light emitting element unit 10 and the light receiving element unit 20 are disposed side by side. A +X direction is a direction from the light emitting element unit 10 toward the light receiving element unit 20. A −X direction is a direction from the light receiving element unit 20 toward the light emitting element unit 10. A Y-axis is an axis orthogonal to the X-axis in the measurement surface 1a. A +Y direction is a direction from a lower side to an upper side in FIG. 2. A −Y direction is a direction from the upper side to the lower side in FIG. 2. A Z-axis is an axis perpendicular to the measurement surface 1a. A +Z direction is a direction from the measurement surface 1a toward the display surface 1b. A −Z direction is a direction from the display surface 1b toward the measurement surface 1a.

The belt 2 is attached to the housing 1 of the measurement apparatus 100. The belt 2 is attached to the housing 1 parallel or substantially parallel to the X-axis. Since the belt 2 is attached to the housing 1 parallel or substantially parallel to the X-axis, the light emitting element unit 10 and the light receiving element unit 20 are disposed in a circumferential direction of the wrist of the user M.

The light emitting element unit 10 emits light toward the measurement site of the user M. The light emitting element unit 10 includes the laser light emitting element 11. The light emitting element unit 10 may include a light emitting element different from the laser light emitting element 11. The light emitting element different from the laser light emitting element 11 is implemented by, for example, a bare chip type or a shell type light emitting diode (LED). The light emitting element unit 10 may include a plurality of laser light emitting elements 11. The number of the laser light emitting elements 11 is appropriately set.

The laser light emitting element 11 emits laser light toward a living body. The laser light emitting element 11 is formed of a semiconductor laser. The laser light emitting element 11 is formed of, for example, a vertical resonant surface light emitting laser. A configuration of the laser light emitting element 11 is appropriately set according to a wavelength range of the emitted laser light. The laser light emitting element 11 emits laser light having a predetermined wavelength in a near infrared region. The wavelength of the emitted laser light is, for example, in a range of 800 nm to 1300 nm. A frequency of the emitted laser light is in a range of 75 THz to 400 THz. The laser light emitting element 11 corresponds to an example of a light emitting unit.

The light receiving element unit 20 receives the scattered light SL scattered by the living body. The light receiving element unit 20 receives the scattered light SL generated when the light emitting element unit 10 emits the laser light to a living body. The light receiving element unit 20 includes the light receiving element 21 that receives the scattered light SL. The light receiving element 21 includes one or more photodiodes. The photodiode is an element in which a photoelectric conversion layer is made of indium gallium arsenide (InGaAs), gallium arsenide (GaAs), silicon, or the like. The photodiode used for the light receiving element 21 is appropriately selected depending on the wavelength of light to be received. The light receiving element 21 corresponds to an example of a light receiving unit.

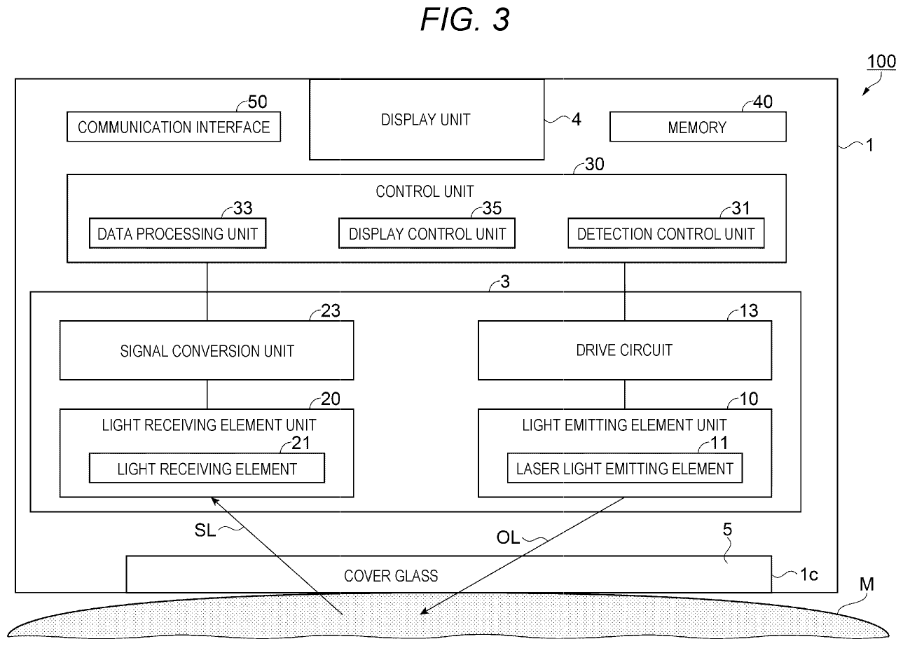

FIG. 3 shows a block configuration of the measurement apparatus 100. FIG. 3 shows the measurement apparatus 100 excluding the belt 2. The measurement apparatus 100 accommodates various units and the like in the housing 1. The measurement apparatus 100 includes the detection unit 3, the control unit 30, the memory 40, the display unit 4, and a communication interface 50. The housing 1 is provided with a housing opening 1c at a position facing the user M. The cover glass 5 is attached to the housing opening 1c.

The cover glass 5 is a protective member that prevents a foreign matter such as dust from entering an inside of the housing 1. The cover glass 5 is attached to the housing 1. The cover glass 5 is provided at a position facing the measurement site of the user M. The cover glass 5 allows the laser light emitted from the laser light emitting element 11 to transmit therethrough. The cover glass 5 allows the scattered light SL generated at the measurement site of the user M to transmit therethrough. The scattered light SL transmitted through the cover glass 5 is received by the light receiving element 21. The cover glass 5 is made of a light transmission material such as glass. At least a part of the cover glass 5 may come into contact with the user M. The cover glass 5 corresponds to an example of a light transmission member.

The detection unit 3 is an optical sensor module that detects data related to biological information measured by using the laser light as a light detection signal. The detection unit 3 includes the light emitting element unit 10, a drive circuit 13, the light receiving element unit 20, and a signal conversion unit 23.

The light emitting element unit 10 emits laser light toward the user M. The light emitting element unit 10 includes the laser light emitting element 11. The light emitting element unit 10 may include a shaping optical system (not shown). The shaping optical system shapes a shape of a spot LS of the laser light. The laser light emitting element 11 emits the laser light to the user M as emitted light OL. The emitted light OL is transmitted through the cover glass 5 attached to the housing opening 1c, so that the user M is irradiated with the emitted light OL.

The drive circuit 13 drives the laser light emitting element 11. The drive circuit 13 causes the laser light emitting element 11 to emit light under the control of the control unit 30. The drive circuit 13 controls a light emission timing, a light emission time, a light emission amount, and the like of the laser light emitting element 11. The drive circuit 13 may control the wavelength of the laser light emitted from the laser light emitting element 11, a size of the spot LS of the laser light, and the like.

The light receiving element unit 20 receives the scattered light SL. The scattered light SL is generated when the light emitting element unit 10 emits the laser light toward the user M. The light receiving element unit 20 receives the reflected light RL reflected by the cover glass 5. The reflected light RL is described later. The light receiving element unit 20 includes the light receiving element 21 and a condenser lens 25 to be described later. The light receiving element 21 receives the reflected light RL and the scattered light SL. The light receiving element 21 receives light and converts the light into an electric signal. The light receiving element 21 transmits the electric signal to the signal conversion unit 23.

The light receiving element 21 receives first component light obtained by the light being diffusely reflected by stationary tissue, and second component light obtained by the light being diffusely reflected by red blood cells moving in a capillary vessel. A first frequency f1, which is a frequency of the first component light, is the same as an emitted light frequency f0, which is a frequency of the emitted light OL emitted from the laser light emitting element 11. The second frequency f2, which is the frequency of the second component light, varies with respect to the emitted light frequency f0 due to the Doppler effect according to the moving speed of the red blood cells. As compared with the emitted light frequency f0, the second frequency f2 fluctuates by a frequency shift amount proportional to the moving speed of the red blood cells. The light receiving element 21 detects a light beat signal reflecting the frequency shift amount.

The signal conversion unit 23 receives the electric signal transmitted from the light receiving element unit 20. The signal conversion unit 23 converts the electric signal into a predetermined light detection signal and transmits the light detection signal to the control unit 30. The light detection signal includes the light beat signal. The signal conversion unit 23 may include an amplification circuit, an extraction circuit, an analog-to-digital conversion circuit, and the like. The amplification circuit amplifies the electric signal. The extraction circuit extracts an AC component from the electric signal. The analog-to-digital conversion circuit converts an analog signal into a digital signal. The light detection signal corresponds to an example of a detection signal.

The control unit 30 is a controller that controls operations of various units. The control unit 30 is, for example, a processor including a central processing unit (CPU). The control unit 30 is implemented by one or more processors. The control unit 30 may be implemented by a field-programmable gate array (FPGA) or an application specific integrated circuit (ASIC). The control unit 30 may include a semiconductor memory such as a random access memory (RAM) or a read only memory (ROM). The semiconductor memory functions as a work area of the control unit 30. The control unit 30 functions as a detection control unit 31, a data processing unit 33, and a display control unit 35 by executing a control program stored in the memory 40. The detection control unit 31, the data processing unit 33, and the display control unit 35 are functional units. The control unit 30 controls the light emitting element unit 10 and the light receiving element unit 20 by each functional unit.

The detection control unit 31 controls the light emitting element unit 10 and the light receiving element unit 20. The detection control unit 31 controls driving of the laser light emitting element 11 via the drive circuit 13. The detection control unit 31 adjusts the light emission timing, an emission intensity, an emission pattern, and the like of the laser light emitting element 11. The detection control unit 31 controls the light receiving element 21. The detection control unit 31 adjusts a light reception timing of the light receiving element 21, an output timing of the electric signal, and the like.

The data processing unit 33 calculates biological information using the light detection signal. The data processing unit 33 performs frequency analysis such as fast Fourier transform on the light detection signal. The data processing unit 33 calculates an intensity spectrum from the light detection signal by performing the frequency analysis. The data processing unit 33 calculates a blood volume, a blood flow, and the like from the intensity spectrum. The blood volume is an index related to the number of red blood cells per unit volume. The blood volume is an index related to a blood volume of the user M. The blood flow is a volume of blood moving in an artery per unit time. The blood flow is an index related to the blood flow of the user M. The data processing unit 33 may calculate a blood pressure or the like using the blood flow. The data processing unit 33 may calculate a pulse or the like using the light detection signal. The data processing unit 33 outputs the biological information such as the blood volume and the blood flow to the display control unit 35. The data processing unit 33 may transmit the biological information such as the blood volume and the blood flow to the memory 40, the communication interface 50, and the like. The data processing unit 33 corresponds to an example of a calculation unit.

The display control unit 35 controls display of the display unit 4. The display control unit 35 causes the display unit 4 to display various images by transmitting display data to the display unit 4. The display control unit 35 acquires the biological information from the data processing unit 33 at a predetermined timing. The display control unit 35 generates display data including the blood volume and the like. The display control unit 35 may generate display data including a blood pressure and the like. The display control unit 35 outputs the display data to the display unit 4. The display control unit 35 causes the display unit 4 to display the biological information based on the display data.

The memory 40 stores various data. The memory 40 stores control data for operating various units, various data measured by the control unit 30, and the like. The memory 40 stores various calculation data used in the data processing unit 33. The memory 40 may store the biological information measured by the data processing unit 33. The memory 40 stores a control program that operates in the control unit 30. The memory 40 includes a ROM, a RAM, and the like.

The communication interface 50 is an interface circuit communicably connected with a tablet terminal 200. The communication interface 50 is connected to the tablet terminal 200 in a wired or wireless manner according to a predetermined protocol. The communication interface 50 includes, for example, a connection port for wired communication, an antenna for wireless communication, and the like. The communication interface 50 receives control data, information related to the user M, and the like from the tablet terminal 200. The communication interface 50 transmits various biological information to the tablet terminal 200. The communication interface 50 may transmit measurement data such as the light detection signal. The communication interface 50 may be communicably connected to an external apparatus other than the tablet terminal 200. The communication interface 50 corresponds to an example of a communication unit.

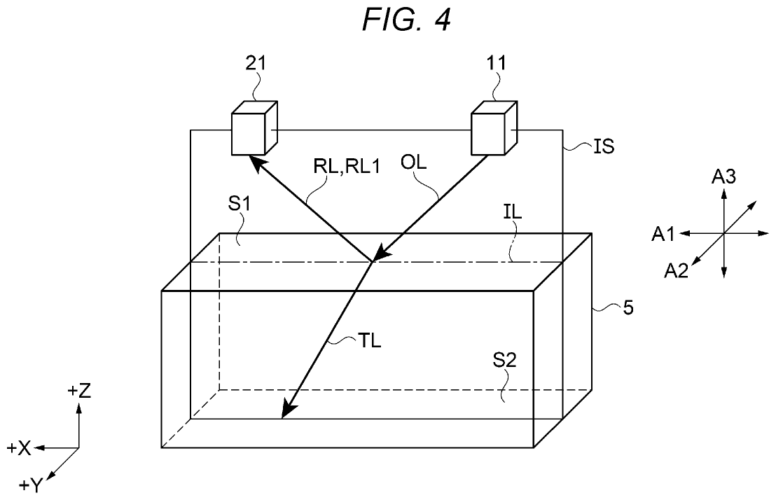

FIG. 4 shows a schematic configuration of the laser light emitted from the light emitting element unit 10. FIG. 4 is a perspective view of the laser light emitting element 11, the light receiving element 21, and the cover glass 5. FIG. 4 shows an entering surface IS. The entering surface IS is a virtual surface on which the laser light emitting element 11 and the light receiving element 21 are disposed. In FIG. 4, the user M is omitted. The measurement site of the user M is located in the −Z direction of the cover glass 5.

The emitted light OL is the laser light emitted from the laser light emitting element 11. The emitted light OL is emitted toward the user M. The emitted light OL enters an inside of the cover glass 5 from a front surface S1 of the cover glass 5. The front surface S1 is a surface where the emitted light OL enters. The front surface S1 is a surface facing the laser light emitting element 11 and the light receiving element 21. The front surface S1 corresponds to an example of a first surface.

The transmitted light TL entering the inside of the cover glass 5 passes through the inside of the cover glass 5. The transmitted light TL that reaches a back surface S2 of the cover glass 5 transmits through the back surface S2, and the measurement site of the user M is irradiated with the transmitted light TL. The back surface S2 is a surface where the transmitted light TL entering the front surface S1 enters. The back surface S2 corresponds to an example of a second surface.

A part of the emitted light OL is reflected by the front surface S1 of the cover glass 5. Front surface reflected light RL1 reflected by the front surface S1 moves toward the light receiving element 21. The front surface reflected light RL1 is an example of the reflected light RL. The front surface reflected light RL1 is received by the light receiving element 21.

The emitted light OL and the front surface reflected light RL1 pass through the entering surface IS. The entering surface IS intersects the front surface S1. The entering surface IS and the front surface S1 intersect at an intersection line IL. Here, a parallel axis parallel to the intersection line IL is referred to as a first optical axis A1. An orthogonal axis orthogonal to the intersection line IL on the front surface S1 is referred to as a second optical axis A2. A vertical axis orthogonal to the front surface S1 is referred to as a third optical axis A3. The first optical axis A1 corresponds to an example of a first axis. The second optical axis A2 corresponds to an example of a second axis.

The first optical axis A1 and the X-axis are disposed in parallel or substantially parallel. The term “substantially parallel” means that a crossing angle between the X-axis and the first optical axis A1 is 5° or less. The laser light emitting element 11 and the light receiving element 21 are disposed along the X-axis and the first optical axis A1. The belt 2 is attached to the housing 1 parallel or substantially parallel to the first optical axis A1. It is preferable that the belt 2 is attached to the housing 1 parallel or substantially parallel to the first optical axis A1. By disposing the laser light emitting element 11 and the light receiving element 21 along the X-axis, the decrease in the measurement accuracy due to a movement of the user M is prevented.

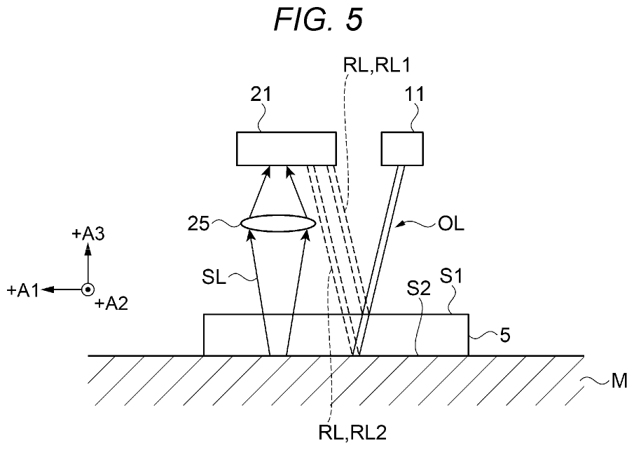

FIG. 5 shows an outline of an optical measurement performed by the detection unit 3. FIG. 5 shows the cover glass 5, the laser light emitting element 11, the light receiving element 21, and the condenser lens 25. FIG. 5 shows the reflected light RL and the scattered light SL when the laser light emitting element 11 emits the emitted light OL toward the user M. FIG. 5 shows the emitted light OL, the reflected light RL, and the scattered light SL on the entering surface IS.

The emitted light OL is emitted toward the measurement site of the user M by the laser light emitting element 11. The emitted light OL enters the cover glass 5. The emitted light OL transmits through the cover glass 5, and the measurement site of the user M is irradiated with the emitted light OL. The emitted light OL is reflected by the front surface S1 and the back surface S2 of the cover glass 5.

The reflected light RL is light obtained by the emitted light OL being reflected by the cover glass 5. The reflected light RL includes the front surface reflected light RL1 and back surface reflected light RL2. The reflected light RL is reflected in a +A3 direction. The +A3 direction is a direction along the third optical axis A3. The +A3 direction is a direction vertically upward with respect to the front surface S1 of the cover glass 5. The reflected light RL is received by the light receiving element 21.

The front surface reflected light RL1 is light obtained by the emitted light OL being reflected by the front surface S1 of the cover glass 5. The front surface reflected light RL1 is generated by a part of the emitted light OL being reflected by the front surface S1. The front surface reflected light RL1 is reflected in the +A3 direction. The front surface reflected light RL1 is received by the light receiving element 21.

The back surface reflected light RL2 is light obtained by the emitted light OL entering the front surface S1 being reflected by the back surface S2 of the cover glass 5. The back surface reflected light RL2 is generated by a part of the emitted light OL entering the front surface S1 being reflected by the back surface S2. The back surface reflected light RL2 is reflected in the +A3 direction. The back surface reflected light RL2 passes through an inside of the cover glass 5. The back surface reflected light RL2 is emitted to the outside from the front surface S1. The back surface reflected light RL2 is received by the light receiving element 21.

The scattered light SL is generated by the measurement site of the user M being irradiated with the emitted light OL passing through the cover glass 5. The scattered light SL is light reflected by living body tissue or red blood cells of the user M. The scattered light SL passes through the inside of the cover glass 5. The scattered light SL is emitted to the outside from the front surface S1. The scattered light SL is condensed by the condenser lens 25. The condensed scattered light SL is received by the light receiving element 21.

The condenser lens 25 condenses the scattered light SL. The condenser lens 25 condenses the scattered light SL to increase a light intensity of the scattered light SL. FIG. 5 shows one condenser lens 25, but the present disclosure is not limited thereto. A plurality of condenser lenses 25 may be provided on an optical path of the scattered light SL.

The light receiving element 21 receives the reflected light RL and the scattered light SL. The light receiving element 21 receives the reflected light RL including the front surface reflected light RL1 and the back surface reflected light RL2. The light receiving element 21 may receive the reflected light RL and the scattered light SL in one light receiving region. The light receiving element 21 may receive the reflected light RL and the scattered light SL in different light receiving regions. The light receiving element 21 generates the electric signal by receiving the reflected light RL and the scattered light SL.

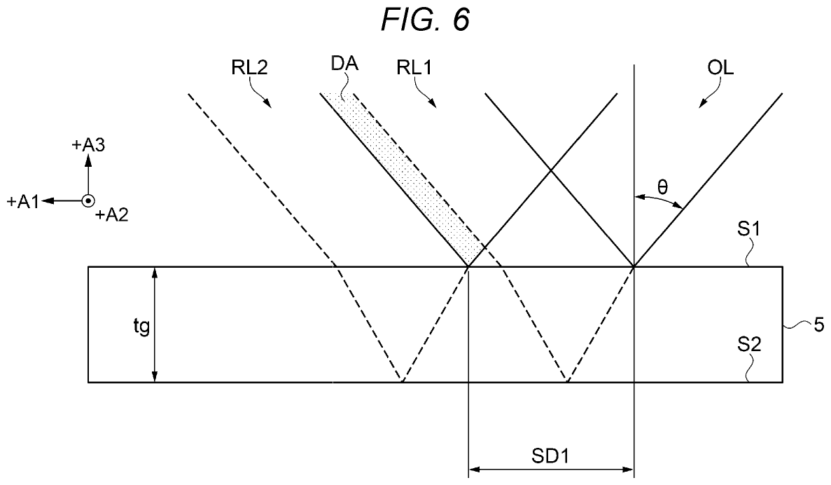

FIG. 6 shows an enlarged configuration of the reflected light RL near the cover glass 5. In FIG. 6, the laser light emitting element 11, the light receiving element 21, the condenser lens 25, the transmitted light TL, and the scattered light SL are omitted. FIG. 6 shows a schematic configuration of the front surface reflected light RL1 and the back surface reflected light RL2 near the cover glass 5. FIG. 6 shows an entering angle θ of the emitted light OL, a glass thickness tg of the cover glass 5, a first spot diameter SD1, and an overlapping area DA.

The entering angle θ of the emitted light OL is an angle at which the emitted light OL enters the front surface S1 of the cover glass 5. The entering angle θ is an angle between the emitted light OL entering the cover glass 5 and the axis parallel to the third optical axis A3. The emitted light OL enters the front surface S1 of the cover glass 5 at the entering angle θ.

The glass thickness ty of the cover glass 5 is a thickness of the cover glass 5 along the third optical axis A3. The glass thickness tg is set in advance. The cover glass 5 is made of a light transmission material having the glass thickness tg and a glass refractive index ng. The glass thickness tg corresponds to an example of a thickness t of the light transmission member. The glass refractive index ng corresponds to an example of a refractive index n of the light transmission member.

The first spot diameter SD1 is a diameter of the emitted light OL along the first optical axis A1. The first spot diameter SD1 is a diameter of the spot LS on the front surface S1 of the cover glass 5 along the first optical axis A1. The first spot diameter SD1 corresponds to an example of a first diameter D1 along the first axis.

The overlapping area DA is an area where the front surface reflected light RL1 and the back surface reflected light RL2 overlap. In the overlapping area DA, interference light of the front surface reflected light RL1 and the back surface reflected light RL2 is generated. The front surface reflected light RL1 and the back surface reflected light RL2 have different phases. Since the front surface reflected light RL1 and the back surface reflected light RL2 overlap each other, interference light having a frequency different from that of the emitted light OL is generated. When the interference light is received by the light receiving element 21, detection accuracy of the light detection signal due to the interference light decreases. The overlapping area DA is preferably set to be narrow.

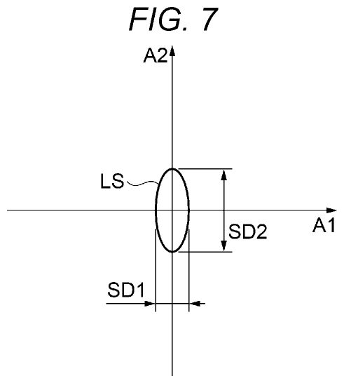

FIG. 7 shows a schematic configuration of the spot LS on the front surface S1 of the cover glass 5. FIG. 7 shows a shape of the spot LS of the laser light on the front surface S1. FIG. 7 shows the first spot diameter SD1 of the spot LS along the first optical axis A1 and a second spot diameter SD2 along the second optical axis A2.

The second spot diameter SD2 is a diameter of the emitted light OL along the second optical axis A2. The second spot diameter SD2 is a diameter of the spot LS on the front surface S1 of the cover glass 5 along the second optical axis A2. The second spot diameter SD2 corresponds to an example of a second diameter D2 along the second axis.

When the first spot diameter SD1 decreases, the overlapping area DA decreases. When the overlapping area DA decreases, the interference light of the front surface reflected light RL1 and the back surface reflected light RL2 decreases. The decrease in the detection accuracy of the light detection signal due to the interference light is prevented. On the other hand, when the first spot diameter SD1 decreases, light amounts of the reflected light RL and the scattered light SL decrease. When the light amounts of the reflected light RL and the scattered light SL decrease, the detection accuracy of the light detection signal decreases due to the decrease in the light amounts. The second spot diameter SD2 does not affect the overlapping area DA. Even when the second spot diameter SD2 increases, the detection accuracy of the light detection signal due to the interference light is unlikely to decrease. Accordingly, the first spot diameter SD1 and the second spot diameter SD2 have a relationship of SD1<SD2. Since the shape of the spot LS is not a circle but a shape in which the first spot diameter SD1 is smaller than the second spot diameter SD2, the decrease in the detection accuracy of the light detection signal due to the interference light is prevented. Since the spot LS has the shape in which the second spot diameter SD2 is larger than the first spot diameter SD1, the decrease in the light amounts of the scattered light SL and the reflected light RL is prevented.

The measurement apparatus 100 includes a laser light emitting element 11 that emits laser light to the user M, a light receiving element 21 that receives the scattered light SL generated when the laser light enters the user M, the housing 1 that accommodates the laser light emitting element 11 and the light receiving element 21, and the cover glass 5 attached to the housing 1 at a position facing the user M. The cover glass 5 has the front surface S1 where the laser light enters and the back surface S2 where the laser light entering the front surface S1 enters. When a parallel axis parallel to the intersection line IL between the entering surface IS on which the laser light emitting element 11 and the light receiving element 21 are disposed and the front surface S1 is defined as the first optical axis A1 and an orthogonal axis orthogonal to the first optical axis A1 on the front surface S1 is defined as the second optical axis A2, the first spot diameter SD1 of the laser light along the first optical axis A1 and the second spot diameter SD2 of the laser light along the second optical axis A2 have a relationship of

SD1<SD2.

Since the shape of the spot LS of the laser light is not a circle but a shape in which the first spot diameter SD1 is smaller than the second spot diameter SD2, the decrease in the detection accuracy of the light detection signal due to the interference light is prevented. Since the spot LS has the shape in which the second spot diameter SD2 is larger than the first spot diameter SD1, the decrease in the light amounts of the scattered light SL and the reflected light RL is prevented.

The measurement apparatus 100 includes the data processing unit 33 that calculates the biological information using the light detection signal obtained when the light receiving element 21 receives the scattered light SL.

The measurement apparatus 100 can obtain the biological information such as the blood volume using the light detection signal.

The measurement apparatus 100 includes the belt 2 that is attached to the housing 1 and is wound around the user M. The belt 2 is attached to the housing 1 parallel or substantially parallel to the first optical axis A1.

By disposing the laser light emitting element 11 and the light receiving element 21 along the X-axis parallel to the first optical axis A1, the decrease in the measurement accuracy due to a movement of the user M is prevented.

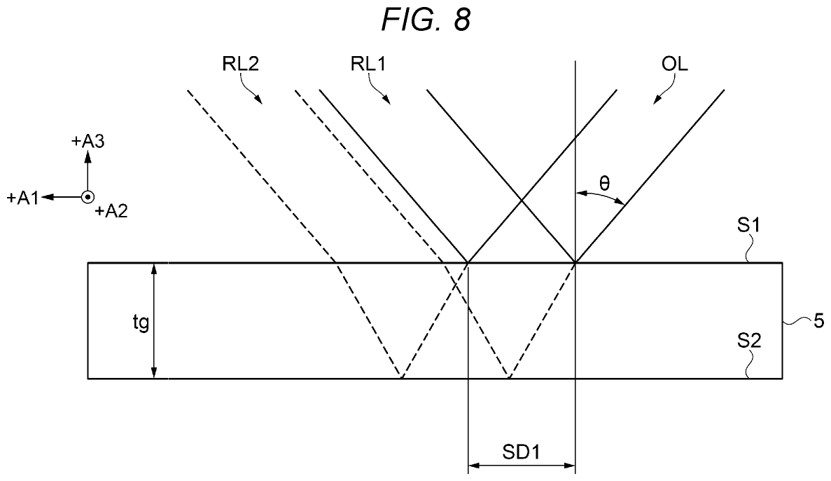

FIG. 8 shows enlarged configuration of the reflected light RL near the cover glass 5. In FIG. 8, the laser light emitting element 11, the light receiving element 21, the condenser lens 25, the transmitted light TL, and the scattered light SL are omitted. FIG. 8 shows a schematic configuration of the front surface reflected light RL1 and the back surface reflected light RL2 near the cover glass 5. FIG. 8 shows the entering angle θ of the emitted light OL, the glass thickness tg of the cover glass 5, and the first spot diameter SD1.

FIG. 8 shows a state in which the first spot diameter SD1 is set to satisfy a relationship of the following Formula.

$$SD 1 < ( tg \times sin θ ) / ng$$

When the first spot diameter SD1 is a diameter having the above relationship, the front surface reflected light RL1 and the back surface reflected light RL2 do not overlap each other as shown in FIG. 8. The overlapping area DA where the front surface reflected light RL1 and the back surface reflected light RL2 overlap is not formed. The interference light between the front surface reflected light RL1 and the back surface reflected light RL2 is prevented from being generated. The decrease in the detection accuracy of the light detection signal due to the interference light of the front surface reflected light RL1 and the back surface reflected light RL2 is further prevented.

It is preferable that first spot diameter SD1, the entering angle θ of the laser light to the front surface S1, the glass thickness tg of the cover glass 5, and the refractive index ng of the cover glass 5 have a relationship of

$$SD 1 < ( tg \times sin θ ) / ng .$$

The decrease in the detection accuracy of the light detection signal due to the interference light of the front surface reflected light RL1 and the back surface reflected light RL2 is further prevented.

### Second Embodiment

A second embodiment shows a system in which the biological information is calculated by the measurement system 1000. The measurement apparatus 100 transmits the electric signal or the light detection signal detected by the light receiving element 21 to the tablet terminal 200. The tablet terminal 200 calculates the biological information using the light detection signal. The tablet terminal 200 analyzes the biological information and displays an analysis result on a display 210.

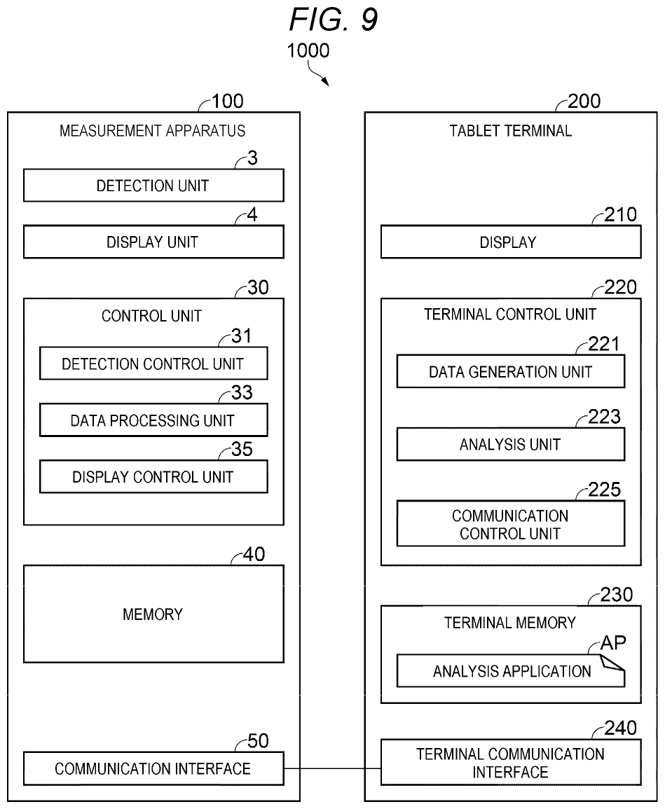

FIG. 9 shows a block configuration of the measurement system 1000. The measurement system 1000 includes the measurement apparatus 100 and the tablet terminal 200. A configuration of the measurement apparatus 100 shown in FIG. 9 is the same as that of the measurement apparatus 100 shown in FIG. 3 except for the function of the data processing unit 33. The laser light emitting element 11 emits the laser light having the relationship of the first spot diameter SD1<the second spot diameter SD2 toward the user M. The laser light emitting element 11 may emit the laser light having the relationship of SD1<(tg×sin θ)/ng toward the user M.

The data processing unit 33 receives the light detection signal transmitted from the signal conversion unit 23. The data processing unit 33 transmits the light detection signal to the tablet terminal 200 via the communication interface 50. The data processing unit 33 transmits the light detection signal to the tablet terminal 200 at a predetermined timing. The data processing unit 33 may transmit a plurality of light detection signals as a light detection signal group to the tablet terminal 200. The data processing unit 33 may receive an electric signal from the signal conversion unit 23 and transmit the electric signal to the tablet terminal 200.

The tablet terminal 200 can calculate the biological information such as the blood volume. The tablet terminal 200 analyzes the biological information. The tablet terminal 200 analyzes a health condition of the user M based on the biological information. The tablet terminal 200 includes the display 210, a terminal control unit 220, a terminal memory 230, and a terminal communication interface 240.

The terminal control unit 220 is a terminal controller that controls operations of various units in the tablet terminal 200. The terminal control unit 220 analyzes the biological information of the user M. The terminal control unit 220 is, for example, a terminal processor including a CPU. The terminal control unit 220 is implemented by one or more processors. The terminal control unit 220 may include a semiconductor memory such as a RAM or a ROM. The semiconductor memory functions as a work area of the terminal control unit 220. The terminal control unit 220 functions as a data generation unit 221, an analysis unit 223, and a communication control unit 225 by executing an analysis application AP stored in the terminal memory 230. The terminal control unit 220 corresponds to an example of an analysis unit.

The data generation unit 221 is a functional unit that operates in the terminal control unit 220. The data generation unit 221 calculates the biological information such as the blood volume. When the data generation unit 221 acquires the light detection signal from the measurement apparatus 100, the data generation unit 221 calculates the biological information using the light detection signal. The data generation unit 221 has the same function as the data processing unit 33 of the first embodiment and calculates the biological information. The data generation unit 221 outputs the biological information to the analysis unit 223.

The analysis unit 223 is a functional unit that operates in the terminal control unit 220. The analysis unit 223 acquires the biological information output from the data generation unit 221. The analysis unit 223 analyzes the biological information to analyze the health condition of the user M. The analysis unit 223 outputs an analysis result of the biological information to the display 210. The analysis unit 223 may store the analysis result in the terminal memory 230. The analysis unit 223 may generate chart data using the biological information. The analysis unit 223 outputs the generated chart data to the display 210. The display 210 displays various charts based on the chart data.

The communication control unit 225 is a functional unit that operates in the terminal control unit 220. The communication control unit 225 controls communication with the measurement apparatus 100. The communication control unit 225 establishes a communication connection with the measurement apparatus 100. The communication control unit 225 causes the measurement apparatus 100 to transmit the light detection signal or the light detection signal group at a predetermined timing. The communication control unit 225 may cause the measurement apparatus 100 to transmit the electric signal at a predetermined timing.

The terminal memory 230 stores various data. The terminal memory 230 stores control data for operating various units in the tablet terminal 200. The terminal memory 230 may store various analysis data analyzed by the terminal control unit 220. The terminal memory 230 stores the analysis application AP operating in the terminal control unit 220.

The analysis application AP operates various functional units by being executed by the terminal control unit 220. The analysis application AP operates the terminal control unit 220 as the data generation unit 221, the analysis unit 223, and the communication control unit 225. The analysis application AP may operate the terminal control unit 220 as a functional unit other than the data generation unit 221, the analysis unit 223, and the communication control unit 225.

The terminal communication interface 240 is a terminal interface circuit communicably connected with the measurement apparatus 100. The terminal communication interface 240 is connected to the measurement apparatus 100 in a wired or wireless manner according to a predetermined protocol. The terminal communication interface 240 includes, for example, a connection port for wired communication, an antenna for wireless communication, and the like. The terminal communication interface 240 receives the light detection signal or the light detection signal group. The terminal communication interface 240 transmits various control data for controlling the operations of the measurement apparatus 100, information related to the user M, and the like to the measurement apparatus 100. The terminal communication interface 240 may be communicably connected to an external apparatus other than the measurement apparatus 100. The terminal communication interface 240 corresponds to an example of a terminal communication unit.

The measurement system 1000 includes a measurement apparatus 100 and a tablet terminal 200. The measurement apparatus 100 includes the laser light emitting element 11 that emits the laser light to the user M, the light receiving element 21 that receives the scattered light SL generated when the laser light enters the user M and generates the light detection signal, the housing 1 that accommodates the laser light emitting element 11 and the light receiving element 21, the cover glass 5 attached to the housing 1 at a position facing the user M, and the communication interface 50 that transmits the light detection signal. The tablet terminal 200 includes the terminal communication interface 240 that receives the light detection signal, and the terminal control unit 220 that analyzes the biological information of the user M using the light detection signal. The cover glass 5 has the front surface S1 where the laser light enters and the back surface S2 where the laser light entering the front surface S1 enters. When a parallel axis parallel to the intersection line IL between the front surface S1 and the entering surface IS on which the laser light emitting element 11 and the light receiving element 21 are disposed is defined as the first optical axis A1, and an orthogonal axis orthogonal to the first optical axis A1 on the entering surface IS is defined as the second optical axis A2, the first spot diameter SD1 of the laser light along the first optical axis A1 and the second spot diameter SD2 of the laser light along the second optical axis A2 have a relationship of SD1<SD2.

Since the shape of the spot LS of the laser light is not a circle but a shape in which the first spot diameter SD1 is smaller than the second spot diameter SD2, the decrease in the detection accuracy of the light detection signal due to the interference light is prevented. Since the spot LS has the shape in which the second spot diameter SD2 is larger than the first spot diameter SD1, the decrease in the light amounts of the scattered light SL and the reflected light RL is prevented.

In the first embodiment and the second embodiment, the cover glass 5 is attached to the housing 1, but the present disclosure is not limited thereto. A detector case surrounding the detection unit 3 may be provided in the housing 1. The cover glass 5 may be attached to the detector case. At this time, the detector case corresponds to an example of the case.

What is claimed is:

1. A biological information measurement apparatus comprising:
a light emitting unit configured to emit laser light to a living body;
a light receiving unit configured to receive scattered light generated when the laser light enters the living body;
a case accommodating the light emitting unit and the light receiving unit; and
a light transmission member attached to the case at a position facing the living body, wherein
the light transmission member has a first surface where the laser light enters and a second surface where the laser light entering the first surface enters, and
D1<D2,
in which a parallel axis parallel to an intersection line between the first surface and an entering surface on which the light emitting unit and the light receiving unit are disposed is defined as a first axis, an orthogonal axis orthogonal to the first axis on the first surface is defined as a second axis, D1 is a first diameter of the laser light along the first axis, and D2 is a second diameter of the laser light along the second axis.

2. The biological information measurement apparatus according to claim 1, wherein
$$D 1 < ( t \times sin θ ) / n ,$$
in which D1 is the first diameter, θ is an entering angle of the laser light to the first surface, t is a thickness of the light transmission member, and n is a refractive index of the light transmission member.

3. The biological information measurement apparatus according to claim 1, further comprising:
a calculation unit configured to calculate biological information using a detection signal obtained by the light receiving unit receiving the scattered light.

4. The biological information measurement apparatus according to claim 1, further comprising:
a band to be attached to the case and wound around the living body, wherein
the band is attached to the case parallel or substantially parallel to the first axis.

5. A biological information measurement system comprising:
a biological information measurement apparatus including a light emitting unit configured to emit laser light to a living body, a light receiving unit configured to receive scattered light generated when the laser light enters the living body and generate a detection signal, a case accommodating the light emitting unit and the light receiving unit, a light transmission member attached to the case at a position facing the living body, and a communication unit configured to transmit the detection signal; and
a control apparatus including a terminal communication unit configured to receive the detection signal, and an analysis unit configured to analyze biological information of the living body using the detection signal, wherein
the light transmission member has a first surface where the laser light enters and a second surface where the laser light entering the first surface enters, and
D1<D2,
in which a parallel axis parallel to an intersection line between the first surface and an entering surface on which the light emitting unit and the light receiving unit are disposed is defined as a first axis, an orthogonal axis orthogonal to the first axis on the first surface is defined as a second axis, D1 is a first diameter of the laser light along the first axis, and D2 is a second diameter of the laser light along the second axis.
